(19) United States
(12) Patent Application Publication
(10) Pub. No.: US 2006/0111420 A1
(43) Pub. Date: May 25, 2006

(54) Sorbicillactone-a derivatives for the treatment of tumour and viral diseases

(75) Inventors: Gerhard Bringmann, Wurzburg (DE); Gerhard Lang, Christchurch (NZ); Jorg Muhlbacher, Freiburg (DE); Karsten Schaumann, Bremerhaven (DE); Stefan Steffens, Aachen (DE); Werner Muller, Weisbaden (DE)

Correspondence Address: SALIWANCHIK LLOYD & SALIWANCHIK;A PROFESSIONAL ASSOCIATION, PO BOX 142950, GAINESVILLE, FL, 32614-2950, US

(73) Assignees: Johannes Gutenberg-Universitat Mainz, Mainz (DE); Freistaat Bayern, Wurzburg (DE)

(21) Appl. No.: 10/525,685

(22) PCT Filed: Jul. 17, 2003
(86) PCT No.: PCT/EP03/07805

§ 371 Date: Jun. 29, 2005

(30) Foreign Application Priority Data
Aug. 21, 2002 (DE) 102 38 257.3

(51) Int. Cl. A61K 31/416; A61K 31/365; C07D 405/02

(52) U.S. Cl. 514/406; 514/469; 548/364.1; 549/307

(57) ABSTRACT

The compounds sorbicillacton A and sorbicillacton-A-derivatives of the general formula I 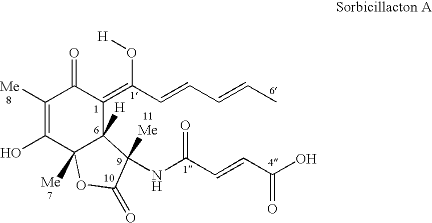 are described, as well as methods for their production. Sorbicillacton A and sorbicillacton-A-derivatives, in cellular culture models, exhibit antitumour- and antiviral properties. Furthermore, sorbicillacton A has inflammation inhibiting properties. Finally, the synthesis of sorbicillacton A and its derivatives is described.

The present invention relates to novel bioactive compounds from marine organisms that are designated as sorbicillacton A, and their derivatives. The invention furthermore relates to a method for producing the compounds, medicaments containing these, and their use in the treatment of diseases. In addition, a synthesis of sorbicillacton A and their derivatives is described.

### BACKGROUND OF THE INVENTION

Marine eukaryotic organisms, in particular sponges, hydrozoes, bryozoes, and tunicates, represent a very rich source of bioactive substances (Sarma A S, Daum T, Müller WEG (1993) Secondary metabolites from marine sponges. Academy of non-profit sciences in Erfurt, Ullstein-Mosby Verlag, Berlin). The reason for this is the fact that these multicellular organisms are sessile organisms that nourish from the microorganisms that are present in their surrounding environment. For this, they need efficient defensive mechanisms in order to protect themselves from bacterial and fungal infections. The most active defensive substances are resembled by those secondary metabolites that are formed by the symbiotes that are present in the marine eukaryotic organisms. Thus, in most cases it remains unclear until today, whether the host (sponges, hydrozoes, bryozoes, and tunicates) or the microorganisms (fungi, bacteria), that are frequently living in a symbiosis with the host, are indeed the producers of the bioactive substances (Althoff et al. (1998) Marine Biol 130:529-536; Wiens et al., Marine Biol.; in press). In recent years, it could also be shown that sponges have defensive mechanisms in order to eliminate viruses (Grebenjuk et al. (2002) Europ. J. Biochem. 269: 1382-1392). Thus, it is a major goal of the research to grow these microorganisms in culture, and to let them produce their bioactive substances there.

Today, it can be assumed that less than 5% of the bacteria and fungi that are present in marine eukaryotic organisms can be held in culture. Their potential is therefore not yet exploited. For a successful and sustainable exploitation of the bioactive potential, it is therefore essential to develop optimal cultivation methods for microorganisms in order to obtain the bioactive substances from the medium with high yields in order to identify these with efficient purification and characterisation methods. Until today, only one medicament has been introduced into the clinical therapy that is produced by marine eukaryotic organisms, the 9-β-D-arabinofuranosyladenosine [araA] (Müller et al. (1977) Ann. New York Acad Sci 284:34-48).

The spectrum of uses for a commercial exploitation of bioactive secondary metabolites is broad and ranges from a treatment of tumours of neurodegenerative diseases to a therapy of infections that are caused by bacteria, viruses and/or fungi.

It is intensively searched for those bioactive substances, that exhibit a high specificity against defined tumours, and, at the same time, reduce opportunistic infections, e.g. by viruses.

It is therefore an object of the present invention to provide a novel bioactive substance from a marine organism a method for its production and its use.

According to the invention this object is first of all solved by providing the bioactive substances that are designated as sorbicillacton A and sorbicillacton-A-derivatives. Sorbicillacton A is a natural compound that has not been described until today. Thus, also its host spectrum is still largely unknown. No substances have been described in the literature that are related to sorbicillacton A.

It was furthermore found that sorbicillacton A and the sorbicillacton-A-derivatives derived therefrom of the general formulae (1) and (2) exhibit pronounced anti-tumour and antiviral properties. Thus, according to the invention, compounds of the general formula (2) are provided: 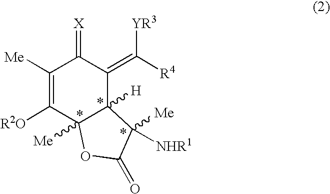
wherein

R¹ is selected from: —H, (C₁-C₁₀)-alkyl, wherein alkyl is straight or branched, (C₃-C₁₀)-alkenyl or an acyl group (e.g. formyl, acetyl, trichloroacetyl, fumaryl, maleyl, succinyl etc.), wherein eventually free —COOH-groups on said acyl group can also be present in form of esters (e.g. a methyl ester, —COOMe) or R¹, optionally, is also one of both heterocyclic acyl substituents (3) or (4)

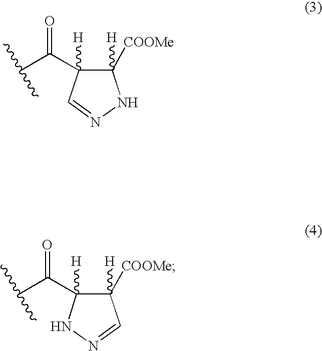

R² is selected from: —H, (C₁-C₁₀)-alkyl, wherein alkyl is straight or branched, or an acyl group (e.g. formyl, acetyl etc.);

R³ is selected from: —H, (C₁-C₁₀)-alkyl, wherein alkyl is straight or branched, or an acyl group (e.g. formyl, acetyl etc.);

R⁴ is selected from: (C₁-C₁₀)-alkyl, wherein alkyl is straight or branched, or (C₃-C₁₀)-alkenyl, wherein the alkenyl residue can contain either one or several double bonds;

X is selected from O, S, NOH or NOR⁵, wherein R⁵ is a straight chain or branched chain (C₁-C₆)-alkyl;

Y either is 0, or Y and X are two N-atoms bound to each other, thus forming a pyrazole ring, and wherein the compound can be present as (R,R,R)-, (R,R,S)-, (R,S,R)-, (R,S,S)-, (S,R,R)-, (S,R,S)-, (S,S,R)- and (S,S,S)-stereoisomer, and pharmaceutically acceptable salts or solvates of (2).

Thereby, preferred according to the invention is a compound having the formula (1): 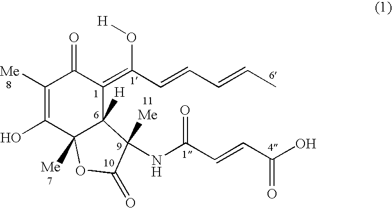
(sorbicillacton A) or derivatives thereof, their diastereomers, as well as the corresponding enantiomers, and pharmaceutically acceptable salts or solvates of this compound.

In the context of the present invention a “derivative” shall be a compound that is derived from the general formula (2), which is, for example, substituted by different residues as given above for R₁ to R₄ and X or Y, as well as mixtures of several of these compounds, which, for example, can be produced into a “personalised” medicament that is matched to a disease to be treated and/or matched to the patients, based on diagnostic data or data with respect to the success of treatment or progression thereof. In addition, a compound of the class of sorbicillacton A shall be understood as a derivative that can be isolated from another (e.g.) marine organism, as those that are mentioned (exemplary) herein.

In the context of the present invention, as a “precursor” of a substance shall be understood, one the one hand, a substance that, during the course of its administration for treatment, is modified in such a way by the conditions inside the body (e.g. pH in the stomach, and the like), or is metabolised after uptake through the body, such that the compound according to the invention or its derivatives are formed as active substances. On the other hand, as precursors derivatives of sorbicillacton A isolated from organisms shall be understood that function as starting material for the synthesis of the compound in the respective organism, and already exhibit the properties of sorbicillacton A as given herein.

It was now found that sorbicillacton A and the sorbicillacton-A-derivatives that are derived from this substance have pronounced and not to be foreseen anti-tumour and anti-viral properties. Furthermore, surprisingly inflammation inhibiting properties of the novel substances could be found. Due to these properties, and based on the finding that sorbicillacton A and the sorbicillacton-A-derivatives are, to a great extent, not toxic for mammals (example mouse), the substances as described herein are suitable for a treatment of tumours and viral diseases. It is recommended to use these substances either in the present form or in form of a depot substance or as a precursor together with a suitable, pharmaceutically acceptable diluent or carrier substance.

A multitude of natural compounds from marine sources, invertebrates and also microorganisms have been described. For the search for novel biologically active compounds, therefore an efficient de-replication is required, that is, known compounds should already be discovered and clearly identified in an early phase of the identification process. For this purpose, the high pressure liquid chromatography (HPLC), coupled with several spectroscopic detection methods, is particularly suited. Here, the LC-triade' is particularly powerful, a coupling of HPLC with NMR, MS, and CD (Bringmann et al. (1998) Anal. Chem. 70:2805-2811; Bringmann et al. (1999) Anal. Chem. 71:2678-2686). The spectroscopic data obtained with respect to ingredient compounds of the extract, without the actual isolation of the individual compounds, allows for the unambiguous identification of already known substances by searching databases. In addition, under suitable circumstances, even the elucidation of the complete stereostructure of unknown natural compounds without prior isolation is possible.

Sorbicillacton A is a natural compound, and sorbicillacton-A-derivatives are synthesis products derived therefrom, that until today were unknown, and whose activity is not described.

According to the invention, these compounds can be processed into tablets, dragées, capsules, drop solutions, suppositories, preparations for injection and infusion with the usual solvents, excipients and carrier substances in order to find therapeutic use for peroral, rectal or parenteral application.

The present invention furthermore relates to a method for producing an above-mentioned compound, that is characterised in that the substance is preferably isolated from a marine organism, such as a fungus of the genus Penicillium growing as a symbiote inside a marine sponge. In the context of the present invention, a fungus of the genus Penicillium (preferably Penicillium chrysogenum) was, for the first time, detected as a producer of the compounds according to the invention. Under the culture conditions as given below, sorbicillacton A is secreted into the culture medium, and is, in addition, accumulated particularly in the fungal biomass. The fungus is a member of the genus Penicillium LNK (1809) which are systematically characterised as anamorph trichocomacae/deuteromycetes/mitosporic fungi, code group 1.A2.15. This genus is characterised by a multitude of wide-spread species that, in part, have considerable biochemical potencies. The present species is a species that is known since 1910 which until today has been described as inhabitant of terrestrial biotopes. The present fungal isolate (fungal strain), nevertheless, is derived from the marine-aquatic environment and was isolated from the marine sponge Ircinia fasciculata (porifera). Based on the structural elucidation as performed in the context of this invention the compound according to the invention can, nevertheless, be produced also by means of common synthesis chemistry, or can be modified into derivatives and precursors. For this, as a further aspect of the present invention, preferred is a method for the biomimetic synthesis of a compound according to the invention, wherein first sorbicillin and/or derivatives thereof is provided, and then an oxidative dearomatisation and subsequent addition of alanin (in case of sorbicillin A) or other amino acids and their analogues (for other derivatives of sorbicillin) is performed in a manner known as such, and a subsequent attachment of fumaric acid (in case of sorbicillin A) or analogous acyl residues (for other derivatives of sorbicillin) is performed.

An additional aspect of the present invention relates to the use of at least one of the above mentioned compounds for the treatment of diseases, such as tumour- and/or viral diseases, and/or for treatment of infectious conditions. This use can be performed, for example, in form of a depot substance or as a precursor, together with a suitable, pharmaceutically acceptable diluent or carrier substance. In case of the treatment of HIV-1 associated diseases, a treatment in a concentration range between 0.3 and 3.0 μg/ml is preferred, in case of the treatment of infections, a concentration of about 2 μg/ml is preferred. In the treatment of the formation of oedema, normally an amount of about 20 μg of the above mentioned compound is used. A further aspect is the use of one or several of the compound(s) according to the invention for the production of a medicament for the treatment of tumour- and/or viral diseases and/or for the treatment of inflammatory conditions. This production can occur in an analogous manner to the one described above and in the above described concentrations and, amongst others, for the above described uses.

A further aspect of the present invention relates to a pharmaceutical composition comprising a compound according to the invention, together with suitable additives or excipients. This pharmaceutical composition can be characterised in that the compound is present in the form of a depot substance or as a precursor together with a suitable pharmaceutically acceptable diluent or carrier substance.

Particularly preferred are pharmaceutical compositions, wherein the compound according to the invention is present in an amount of 20 μg (particularly suitable for the treatment of formation of oedema) or pharmaceutical compositions, wherein the compound according to the invention is present in such an amount that a concentration range between 0.3 and 3.0 μg/ml is present during the treatment in vivo (particularly suitable for the treatment of viral and/or inflammatory disease).

Preferred is a pharmaceutical composition according to the present invention that contains additional chemotherapeuticals. These chemotherapeuticals can comprise all chemotherapeuticals that are common for the person of skill in the context of a cancer therapy (e.g. taxol or others).

In accordance with the invention, the above mentioned pharmaceutical composition can be present in the form of tablets, dragées, capsules, droplets, suppositories, preparations for injection or infusion for a peroral, rectal or parenteral use. Such administration forms and their production are known to the person of skill.

The products of the methods of the general formula (1) and (2) exhibit valuable pharmacological properties. The antitumour effect was confirmed using, amongst others, the L5178y-mouse lymphoma cellular system (ATCC CRL 1722). These cells were held in suspension culture, such as already described earlier (Müller et al. (1979) Cancer Res. 39: 1102-1107). The ED₅₀-concentrations for sorbicillacton A (inoculation: 10.000 cells/ml; time of incubation: 72 hrs.) in these tumour-cell strains were found at 2.2±0.3 μg/ml. Sorbicillacton A was slightly lower effective in the tumour cell lines PC-12 (adrenal, phaeochromocytomal tumour [rat]; ATCC CRL 1721), Sarcoma 180 (mouse-sarcoma; ATCC TIB 66) and HeLa S3 (epitheloid carcinoma [cervix; human]; ATCC CCL 2.2) with ED₅₀-concentrations between 8 and 15 μg/ml.

The present invention furthermore relates to a method for treatment of a disease selected from tumour- and/or viral diseases and/or inflammatory conditions, comprising the administration of a compound according to the invention, such as in the form of a pharmaceutical composition according to the invention. According to the invention, the administration can occur in the form of a depot substance or as precursor, together with a suitable, pharmaceutically acceptable diluent or carrier substance.

Particularly preferred is a method for treatment, wherein the viral disease is a HIV-1-infection. Thereby, the administration of the compound can take place in a concentration range in vivo of between 0.3 and 3.0 μg/ml. The amounts that are required in order to achieve these concentrations are readily derivable for the person of skill, which, amongst others, depend from the respective patient, the disease, and the bioavailability of the respective compound to be used. As additional viral diseases to be treated, exemplary additional HIV-infections, infections with HCV (hepatitis C-virus), herpes and/or RSV-viral diseases shall be given.

Further preferred is a method for the treatment of a disease, wherein an inflammation is treated. For this, the compound can be administered analogously as for the administration in viral diseases in a concentration in vivo of 2 μg/ml. A further treatment being possible is the therapy of the formation of an oedema. For this, a compound according to the invention can be administered in an amount of 20 μg.

Of particular importance for the use of the products of the methods for a chemotherapy of tumour diseases, and also for the antiviral therapy, is the fact that a cytostatic effectiveness on non-tumour cells in culture is lacking at the concentrations to be used in therapy. This result was concluded from experiments with lymphocyte cultures: Splenal lymphocytes were obtained from six weeks old NMRI-mice; the erythrocytes were removed from the suspension by treatment with ammonium chloride. The splenal lymphocytes were held in RPMI 1640-medium with 20% foetal calf serum in a density of 1.5×10⁷ cells/ml for 72 hrs. in the presence of 2 μg/ml concanavalin A. 18 Hrs. before the end of the experiment, [³H]-thymidin was added. In case of an incubation with 15 μg/ml of the products of the method, no adverse effect on the rate of DNA-synthesis was measured. In the presence of 20 μg/ml, only a 20% inhibition of the integration rate of [³H]-thymidin into the DNA took place.

The sub-acute toxicity of sorbicillacton A and its derivatives in a treatment of mice over five days i.p.- with values at >>20 mg/kg is so advantageous, that a use of the products of the method in a chemotherapy of cancerous diseases is promising.

The pronounced antiviral effectiveness of the products of the method was confirmed in the HTLV-IIIB (HIV-1) test system. A specific inhibition of the viral production was detected at a concentration range of between 0.3 and 3.0 μg/ml.

In addition, sorbicillacton A and sorbicillacton-A-derivatives develop pronounced inflammation inhibiting properties. These effects could be measured both in vitro (model: inhibition of the phospholipase A2 [from bee's poison]) and in vivo (model: mice-ear oedema). It could be shown in in-vitro-experiments with phospholipase A2 (from bee's poison) that at a concentration of 2 μg/ml a nearly 80% inhibition could be obtained. Furthermore, the effect of sorbicillacton-A on the mouse oedema was measured. In the mice (Swiss; about 25 g), the oedema were induced with TPA (10 μg). TPA was dissolved in acetone and applied topically onto the right inner auricle. The left inner auricle served as a control (acetone-control). The animals were sacrificed after 4 hours by cervical dislocalisation, and the areas of oedema were cut out. These were subsequently weighted. The ratio between the weight of the treated tissue to the control was used as a measure for the effect of sorbicillacton-A (Carlson R P et al. (1985) Agents Actions 17: 197-204). Usually, the active substance was topically applied 10 min after the TPA-treatment onto the treated site. After 3 hours, the controls developed an oedema of 12.3±0,9 mg. The effect of sorbicillacton A on the formation of the oedema was significant (P<=0.01); in a treatment with 20 μg, a 39.2±5.3% inhibition was obtained (n=5).

The invention shall now be further illustrated in the following based on examples, without being limited to these examples in any way.

### EXAMPLE 1

### Obtaining the Substance Sorbicillacton A from Biological Material

For the first time, a fungus of the genus Penicillium (preferably Penicillium chrysogenum) was detected as a producer of the of the novel natural compound sorbicillacton A. Under the culture conditions as given below, sorbicillacton A is secreted into the culture medium, and in addition, is particularly accumulated in the fungal biomass. The fungus is a member of the genus Penicillium LINK (1809) which are systematically characterised as anamorph trichocomacae/deuteromycetes/mitosporic fungi, code group 1.A2.15. This genus is characterised by a multitude of wide-spread species that, in part, have considerable biochemical potencies. The present species is a species that is known since 1910 that has been described until today as inhabitant of terrestrial biotopes. The present fungal isolate (fungal strain), nevertheless, is derived from the marine-aquatic environment and was isolated from the marine sponge Ircinia fasciculata (porifera).

Description of the General Methods for Isolating and Culturing of the Fungus

Subsequently, the culture broth including the grown mycelium is harvested, supplemented with 40 ml ethyl acetate per 300 ml culture broth, and deep-frosted at −86° C.

For extraction, preferably methanol, dichloromethane, and acetic acid ester is used, nevertheless, also other solvents, such as ethanol, propanol, butanol, ether, n-hexane, benzene, toluene, acetone, methylethylketone, acetic acid-tertiary butylester are conceivable. The obtained extracts are concentrated in vacuo until dryness, and were separated, optionally after pre-fractionalisation, by liquid-liquid-extraction with the aid of one or several chromatographic methods. For this, preferably the preparative HPLC on ‘reversed-phase’-material (RP₁₈) with a water/acetonitrile or a water/methanol gradient is used. Siliciumdioxide, aluminiumoxide or cellulose can also find use as stationary phases, or liquid-liquid-chromatography, e.g. HSCCC, could be employed. Different fractions are collected, and examined by HPLC or thin-layer chromatography for their content of the compounds according to the invention. After concentration of the fractions in question, the compound is obtained in pure form.

### EXAMPLE 2

### Examples for the Compounds According to the Invention

Isolation of sorbicillacton A

The present fungal strain of Penicillium was isolated on 02.05.2001 from the marine sponge Ircinia fasciculata from 17.5 m sounding in the bay of Fetovaia (42°43′24″N/10°09′31″E) on Elba, Italy. Immediately after harvesting, the sponge was examined for its fungal content with the aid of respective marine-mykologic methods. The present isolation was obtained by laying out of small pieces of tissue of the dissected sponge onto a nutrient agar plate of the following composition (CYAS, according to Pitt 1973):

Czapek-yeast extract-agar+seawater:
30 g sucrose
5 g yeast extract
3 g NaNO₃
1 g K₂HPO₄
10 ml mineral-solution:
5 g KCl, 5 g MgSO₄+7H₂O, 0.1 g FeSO₄+7H₂O/100 ml H₂O 1 ml trace metal-solution:
1 g ZnSO₄+7H₂O, 0.5 g CuSO₄+5H₂O/100 ml H₂O antibiotics
1000 ml seawater (30-33 PSU)

The crude primary culture was further grown by several purification steps into an axenic pure culture. The stock culture was performed on slant agar tubes of the following composition (GPYNS, Schaumann 1974):

Glucose-peptone-yeast extract-ammonium nitrate-seawater-agar:
1.0 g glucose
0.5 g peptone
0.1 g yeast extract
1.0 g ammonium nitrate
15.0 g agar
1000 ml seawater (30-33 PSU)
P_{H} 7.2-7.4

The growth of the fungal culture for obtaining the novel natural compound sorbicillacton A was performed in 1-1-Erlenmeyer beakers, that were each filled with 300 ml nutrient solution of the following composition (WS, according to Wickerham 1951):

Wickerham-seawater-medium:
3.0 g yeast extract
3.0 g malt extract
5.0 g peptone
10.0 g glucose
1000 ml seawater (30-33 PSU)
P_{H} 7.2-7.4

The sterilisation of the nutrient solution takes place by autoclaving at 121° C./1 bar, 15 minutes. As inoculum for the experimental fungal culture, ten pieces of slices of mycelium (diameter 5 mm) of each beaker were used. These were punched out from a 7 days old preculture on WS-agar with the aid of a cork drill, and transferred into the nutrient solution. The incubation of the inoculated beakers took place over a period of 14 days at room temperature or also at constant 20° C. in static culture in the dark. Subsequently, the grown mycelium including the culture broth is harvested, supplemented with 40 ml ethyl acetate per 300 ml culture broth, and deep-frozen at −86° C.

In three 300-ml-culture preparations, the mycelium was separated from the culture medium by filtration, reduced into small pieces, and extracted with 250 ml of a dichloromethane-methanol-mixture (1.1) under stirring for 48 h. Subsequently, the mycelium was separated by centrifugation, and the extract was concentrated in vacuo until dryness. The culture filtrate was extracted three times each with 250 ml acetic acid ester, the acetic acid ester phases were combined, and also concentrated in vacuo until dryness. Culture filtrate and mycelium extract were dissolved together in a mixture of 200 ml methanol and 6 ml water, and extracted with 200 ml petrol ether. The petrol ether phase was discarded, the methanol-water-phase was concentrated in vacuo, and examined by means of HPLC-UV, -NMR and -MS. By comparison of the so-obtained spectroscopic data of different ingredients with databases, several known compounds could be identified, e.g. meleagrin and roquefortin C. One of the compounds was recognised as novel, yet unknown, natural compound. Since two-dimensional HPLC-NMR-experiments on the extract, e.g. HPLC-WET-COSY and HPLC-WET-ROESY, showed that it concerned a highly interesting structure, this compound was isolated by preparative HPLC:
Column: Waters SymmetryPrep C18, 19×300 mm
Eluent: acetonitrile+0.05% TFA, Water+0.05% TFA
Gradient: from 10% acetonitrile to 100% acetonitrile in 30 min
Flow: 11 ml/min
Detection: 254 nm

Sorbicillacton A eluated between 19 and 20 min. The corresponding fractions were collected and concentrated in vacuo until dryness. 6 mg of a yellow, amorphic solid were obtained being soluble in methanol.

Structure Elucidation of the Compound

The sorbicillacton A as obtained has the following spectroscopic properties, summarised in table 1:

TABLE 1
NMR-data of sorbicillacton A (approx.
6 mg in THF-d₈; 600 MHz)

| | ¹³C | ¹H | | | |
|---|---|---|---|---|---|
| Position | [ppm] | [ppm] | COSY | HMBC | ROESY |
| 1 | 99.55 | | | | |
| 2 | 192.10 | | | | |
| 3 | 110.92 | | | | |
| 4 | 166.53 | | | | |
| 5 | 80.98 | | | | |
| 6 | 53.00 | 3.43, s | | 1, 2, 4, 5, | 7, 11, |
| | | | | 7, 9, 11, 1′ | 2′ |
| 7 | ˜25.00 | 1.55, s | | 5, 6 | 6 |
| 8 | 7.29 | 1.54, s | | 2, 3, 4 | |
| 9 | 59.98 | | | | |
| 10 | 172.98 | | | | |
| 11 | ˜26.00 | 1.42, s | | 6, 9, 10 | NH, 6, |
| | | | | | 2′ |
| 1′ | 169.72 | | | | |
| 2′ | 121.68 | 6.38, d | 3′ (14, 7 Hz) | 1′, 4′ | 6, 11 |
| 3′ | 139.12 | 7.19, dd | 2′, 4′ (11 Hz) | 4′, 5′ | 5′ |
| 4′ | 131.95 | 6.28, ddd | 3′, 5′, 6′ | 6′ | |
| | | | (1.3 Hz) | | |
| 5′ | 136.91 | 6.08, m | 4′ (14.5 Hz), | 3′, 6′ | 3′ |
| | | | 6′ (6.2 Hz) | | |
| 6′ | 18.54 | 1.83, dd | 4′, 5′ | 4′, 5′ | |
| 1″ | 162.53 | | | | |
| 2″ | 136.01 | 6.67, d | 3″ (15.4 Hz) | 1″, 3″, 4″ | NH |
| 3″ | 131.22 | 6.49, d | 2″ | 1″, 2″, 4″ | |
| 4″ | 166.31 | | | | |
| 1′-OH | | 16.60, s | | 1, 1′, 2′ | |
| NH | | 7.60, s | | 9, 10, 11, 1″ | 11, 2″ |

ESI-MS (in MeCN/H₂O): m/z 418 [M + H]⁺, 459 [M + MeCN + H]⁺
FAB-MS (in 3-nitrobenzyl alcohol): m/z 418 [M + H]⁺
UV/VIS (in acetonitrile/water + 0.05% TFA): λₘₐₓ [nm] 215, 271, 379

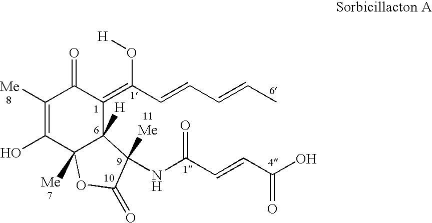

The isolated compound, a yellow, amorphic solid, showed an [M+H]⁺-signal in the ESI-MS at m/z=418, the molecular mass of 417 as resulting therefrom is also confirmed by the FAB-MS-measurement.

From the NMR-data, in particular the HMBC— and COSY-correlations, two partial structures of the molecule can be derived. A fumaric residue (5), wherein the E-configuration of the double binding is occupied by the high coupling constant (15.4 Hz), and a sorbyl residue (6). How the latter is bound to the residue of the molecule remains unclear for the time being, the chemical shift (169.7 ppm), measured for C-1′, allows for both the possibilities of a sorbine acid ester as well as a C—C-bound sorbyl residue in the enol form. The substitution pattern of the central 6-ring (7) is available via HMBC-interactions of the three methyl groups C-7, C-8 and C-11 as well as from H-6.

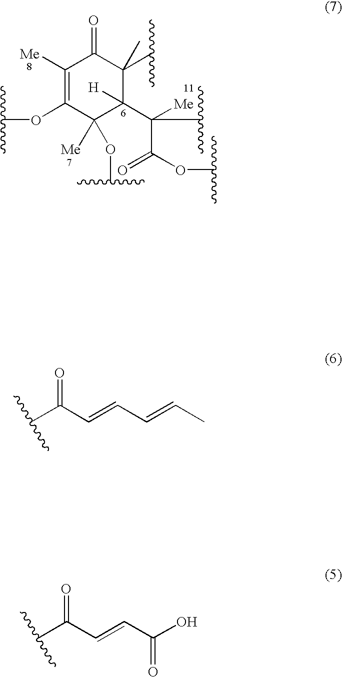

In addition, performing the NMR-measurements in THF-d₈ allows for observing interchangeable protons and their interactions. Hereby, one recognises two additional sharp signals in the ¹H-spectrum, an enolic hydroxy group that is strongly shifted to a deep field (16.6 ppm) by the hydrogen bond formation to the β-attached keto group in the ring, and an amidic proton at 7.6 ppm. Based on the HMBC-correlations of these protons, all three partial structures can be combined into a total structure (8).

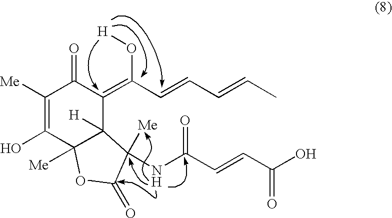

For assuring the position of the lactone ring and the free acid and hydroxy groups, the natural compound was methylated with diazomethane, whereby a dimethylated derivative was formed, wherein additionally a cyclo-addition of diazomethane at the double bound of the fumarate residue had taken place. NMR-measurements on this derivative led to the structure (9) [sorbicillacton-A-derivative 1 (SOA-D1)], whereby the structure (1) is confirmed for the natural compound. The relative configuration of the three stereocentres can be determined by ROESY-interactions: The correlations of H-6 to the methyl groups 7 and 11 confirm that both are cis-positioned to H-6.

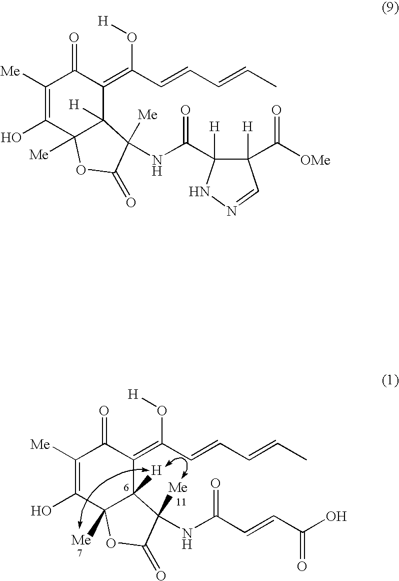

In addition, the absolute configuration of the novel natural compounds could be elucidated. Since the present concerns a completely novel type of structure, an assignment by comparison of the CD-spectrum with the structurally related configuratively known substance could not simply be done. The assignment, nevertheless, could readily be achieved by using modern quantum chemical CD-calculations, based on simulation of CD-spectra to be expected for both enantiomers in question, and comparing these spectra as predicted by calculation with the actual spectrum that was experimentally measured for the natural compound. Using this strategy, the complete absolute stereostructure of sorbicillacton A as illustrated in the figure was established.

### EXAMPLE 2

### Derivatisation of Sorbicillacton A to Pyrazole Derivatives

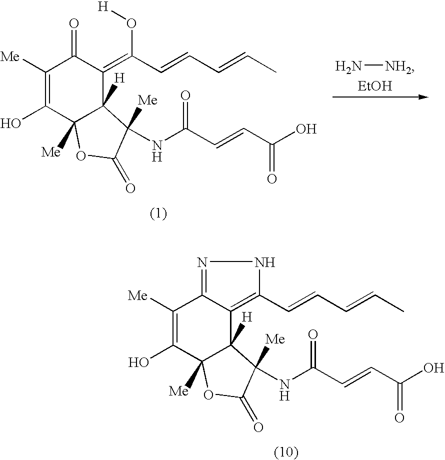

To a solution of 100 mg sorbicillacton A (1) in 2 ml Ethanol, 10 mg hydrazine is added. After stirring for 6 h at room temperature, the solvent was evaporated. The purification of the residue occurs by means of column chromatography on silica gel (solvent: mixture of dichloro-methan-methanol), and results into the desired pyrazole derivative (10).

### EXAMPLE 3

### Biological Properties of the Compounds

a) Antitumoural Activity

The antitumoural activity of sorbicillacton A and one of its derivatives (exemplary shown here using derivative SOA-D 1) was tested on a series of tumour-transformed cells, such as the L5178y mouse lymphoma cellular system (ATCC CRL 1722). As described (Müller et al. (1979) Cancer Res. 39: 1102-1107), the cells were cultured in RPMI-medium, to which 10% foetal calf serum was added. 10.000 cells/ml were chosen as inoculum concentration. At the starting point the chosen substance was added, and the culture was incubated for 72 h. Thereafter, the number of living cells was determined by means of the colorimetrc XTT-appoach, and analysed with an ELISA reader (see: Scudiero D A, Shoemaker R H, Paull K D, Monks A, Tierney S, Nofziger T H, Currens M J, Seniff D, Boyd M R (1988) Evaluation of a tetra-zolium/formazan assay for cell growth and drug sensitivity in culture using human and other tumour cell lines. Cancer Res 48: 4827-4833; Daum T, Engels J, Mag M, Muth J, Lücking S, Schröder HC, Matthes E, Müller WEG (1992) Antisense oligonucleotides: inhibitors of splicing of mRNA of human immunodeficiency virus. Intervirology 33: 65-75). The optical density of the controls was defined as 100%.

| | Concentration of the | Optical density |
|---|---|---|
| Compound | compound (μg/ml) | (595 nm) |
| Control | 0 | 0.38 |
| Sorbicillacton A | 0.1 | 0.24 |
| | 0.3 | 0.19 |
| | 1.0 | 0.07 |
| | 3.0 | 0.06 |
| SOA-D1 | 0.1 | 0.29 |
| | 0.3 | 0.22 |
| | 1.0 | 0.05 |
| | 3.0 | 0.03 |

Result: It becomes clear that, at the low concentration of >0.1 μg/ml of sorbicillacton A and derivative SOA-D1, the cellular proliferation was drastically reduced after 72 h. The ED50-concentration (calculated according to Sachs L. (1984) Applied Statistics. Springer, Berlin) for the L5178y cells used was found at the 0.18 μg/ml.

In addition to the tumour-transformed cells, the influence of the products of the method was also examined on human foreskin-fibroblasts. The cells and the method for cultivation were described earlier (see: Müller WEG, Maidhof A, Zahn R K, Schröder HC, Gasic M J, Heidemann D, Bernd A, Kurelec B, Eich E, Seibert G (1985) Potent antileukemic activity of the novel cytostatic agent avarone and its analogues in vitro and in vivo. Cancer Res. 45: 4822-4827). Cells between the 6. and 9. passage were used for the experiments. The cultivation was performed in collagen-coated plastic flasks. The cellular count was determined microscopically. The experients show that, at a concentration of 30 μg/ml, the sorbicillacton A exerted no effect on the proliferation.

b) Antiviral Activity

An extensive listing of the references and the performance of the methods is summarised in earlier publications (Sarin P S, Sun D, Thornton A, Müller WEG (1987) Inhibition of replication of the etiologic agent of acquired immune deficiency syndrome (human T-lymphotropic retrovirus/lymphadenopathy-associated virus) by avarol and avarone. J Natl Cancer Inst 78: 663-666; Schröder HC, Sarin P S, Rottmann M, Wenger R, Maidhof A, Renneisen K, Müller WEG (1988) Differential modulation of host cell and HIV gene expression by combinations of avarol and AZT in vitro. Biochem Pharmacol 37: 3947-3952).

Examination Parameter: Cellular Growth

H9-cells as well as H9-cells infected with HTLV-IIIB (HIV-1) were used for the inoculation of a culture mediums in a concentration of 0.2×1,000,000 cells/ml culture medium. After 4 days of incubation, the density of the H9-cells was 1.3×1,000,000 cells/ml, whilst the density of the H9-cells infected with HTLV-IIIB was only 0.6×1,000,000 cells/ml, both these values formed the control values.

Then, the samples of H9-HTLV-III-cells (0.2×1,000,000 cells/ml) were treated with different concentrations of sorbicillacton A for 4 days. The following results were obtained:

| | Concentration of the | Cellular concentration × |
|---|---|---|
| Compound | compound (μg/ml) | 1,000,000/ml |
| control | 0 | 0.62 |
| sorbicillacton A | 0.1 | 0.68 |
| | 0.3 | 0.83 |
| | 1.0 | 1.39 |
| | 3.0 | 0.92 |

Result: It can be seen that sorbicillacton A, in the concentrations between 0.3 and 3.0 μg/ml, increases the growth rate of H9-HTLV-IIIB-cells to values that are located in the range of the controls, i.e. H9-cells without HTLV-IIIB.

Examination Parameter: Production of Reverse Transcriptase

In this approach it is tested, to which extent the products of the method inhibit the production of HIV(HTLV-IIIB)-viruses in H9-cells. The reverse transcriptase as present in these particles was measured as a parameter for the amount of viruses.

It was examined, to which extent the production of HTLV-IIIB (HIV-1)-viruses is reduced after a 4-day gavage of sorbicillacton A or the sorbicillacton-A-derivative to H9-HTLV-IIIB-cells. As a measure of the viral amount in the culture medium, the reverse transcriptase was chosen, i.e. the inhibition of the reverse-transcriptase-production indicated the inhibition of the viral production. The results are summarised in the following table:

| | concentration of the | reverse transcriptase activity |
|---|---|---|
| Compound | compound (μg/ml) | (100%) |
| control | 0 | 100 |
| sorbicillacton A | 0.1 | 83 |
| | 0.3 | 28 |
| | 1.0 | 22 |
| | 3.0 | 14 |

Result: It can be seen that in the supernatant of the H9-HTLV-IIIB-cells that were not treated with sorbicillacton A, a considerable activity of the reverse transcriptase was present. The addition of sorbicillacton A or the sorbicillacton-A-derivative results in a dose-dependent reduction of the activity of reverse transcriptase in the supernatant. A considerable inhibition was observed already at a dosage of 0.1 μg/ml. The compounds for use according to the invention thus have the ability to nearly completely inhibit viral replication in concentrations wherein different in-vitro-parameters, for example the cellular growth, practically can not be influenced.

Examination Parameter: Expression of the p24- and p15-Proteins

It could be shown that sorbicillacton A and the sorbicillacton-A-derivative SOA-D1 possessed a strong inhibiting effect on the expression of HIV p24 (gag-protein) and p15 (Gag-Protein) in infected H9-cells. When the target-H9-cells were grown with the HIV(HTLV-IIIB)-isolate and without the compound to be tested, an expression of the p24- and p15-proteins took place, as could be confirmed by means of indirect immunofluorescence-assays. Following incubation of the H9-HTLV-IIIB-cells with the compounds to be tested, however, an essentially complete protective effect was observed. The expression of the p24- and p15-proteins was reduced up to 24%. The following results were obtained:

| | | Expression of p15 and p24 | |
|---|---|---|---|
| | Concentration of the | (in %) | |
| Compound | compound (μg/ml) | p15 | p24 |
| control | 0 | 100 | 100 |
| sorbicillacton A | 0.1 | 98 | 93 |
| | 0.3 | 65 | 42 |
| | 1.0 | 31 | 28 |
| | 3.0 | 47 | 24 |

Result: It can be seen that sorbicillacton A and the sorbicillacton-A-derivative (SOA-D1) lead to a significant reduction of the expression of the HTLV-IIIB (HIV-1)-proteins.

### EXAMPLE 4

### Effect of Sorbicillacton a In Vivo

For these examinations, male (outbred) NMRI-mice (32-36 g; age: 8-9 months) were used. The test substance sorbicillacton A was dissolved in methyl cellulose, and injected into the animals i.p. A dosage of 20 mg/kg (per day) was administered to the animals for five days. After the treatment, the weight of the animals was determined. During this time the weight of the sorbicillacton-A-treated animals (33±4 g) did not differ significantly from those of the controls [not treated with sorbicillacton A] (35±4 g). None of the test animals dies.

Result: It is concluded from this data that the subacute toxicity of sorbicillacton A in a five-day i.p.-treatment is >>20 mg/kg.

Description of the Synthesis of Sorbicillacton A and their Derivatives

Sorbicillacton A and a whole series of structural analogues can be produced in a few steps by biomimetic synthesis starting from sorbicillin and related compounds, by oxidative dearomatisation and subsequent addition of alanin (in case of sorbicillacton A) or other amino acids and their analogues, and subsequent attachment of fumaric acid (in case of sorbicillacton A) or analogue acyl residues.

1-23. (canceled)

24. A compound of the general formula (2): 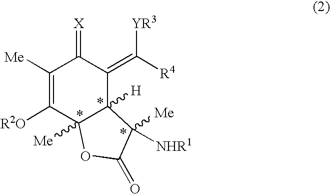
wherein
R¹ is selected from the group consisting of: —H, (C₁-C₁₀)-alkyl (wherein alkyl is straight or branched), (C₃-C₁₀)-alkenyl, and acyl groups, wherein free —COOH-groups can be present on the acyl group in the form of esters; or, optionally, R¹ can be (3) or (4) 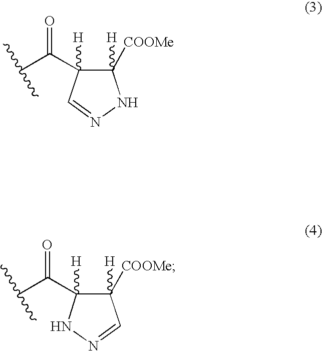
R² is selected from the group consisting of: —H, (C₁-C₁₀)-alkyl (wherein alkyl is straight or branched), and acyl groups;
R³ is selected from the group consisting of: —H, (C₁-C₁₀)-alkyl (wherein alkyl is straight or branched), and acyl groups;
R⁴ is selected from the group consisting of: (C₁-C₁₀)-alkyl (wherein alkyl is straight or branched), and (C₃-C₁₀)-alkenyl, wherein the alkenyl residue can contain one or more double bonds;
X is selected from the group consistinf of O, S, NOH and NOR⁵, wherein R⁵ is a straight chain or branched chain (C₁-C₆)-alkyl;
Y is O, or Y and X are N-atoms bound to each other thus forming a pyrazole ring;
wherein the compound can be present as an (R,R,R)-, (R,R,S)-, (R,S,R)-, (R,S,S)-, (S,R,R)-, (S,R,S)-, (S,S,R)- or (S,S,S)-stereoisomer; and pharmaceutically acceptable salts or solvates of (2).

25. The compound according to claim 24 having the formula (1): 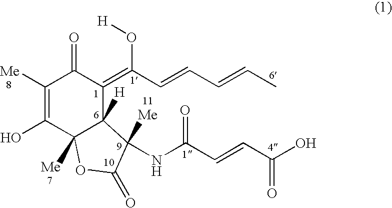
(sorbicillacton A) and derivatives thereof, their diastereomers, as well as the corresponding enantiomers, and pharmaceutically acceptable salts or solvates of this compound.

26. A method for the production of a compound of the general formula (2): 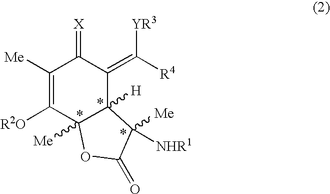
wherein
R¹ is selected from the group consisting of: —H, (C₁-C₁₀)-alkyl (wherein alkyl is straight or branched), (C₃-C₁₀)-alkenyl, and acyl groups, wherein free —COOH-groups can be present on the acyl group in the form of esters; or, optionally, R¹ can be (3) or (4) 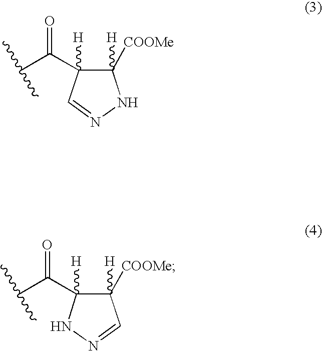
R² is selected from the group consisting of: —H, (C₁-C₁₀)-alkyl (wherein alkyl is straight or branched), and acyl groups;
R³ is selected from the group consisting of: —H, (C₁-C₁₀)-alkyl (wherein alkyl is straight or branched), and acyl groups;
R⁴ is selected from the group consisting of: (C₁-C₁₀)-alkyl (wherein alkyl is straight or branched), and (C₃-C₁₀)-alkenyl, wherein the alkenyl residue can contain one or more double bonds;
X is selected from the group consistinf of O, S, NOH and NOR⁵, wherein R⁵ is a straight chain or branched chain (C₁-C₆)-alkyl;
Y is O, or Y and X are N-atoms bound to each other thus forming a pyrazole ring;
wherein the compound can be present as an (R,R,R)-, (R,R,S)-, (R,S,R)-, (R,S,S)-, (S,R,R)-, (S,R,S)-, (S,S,R)- or (S,S,S)-stereoisomer; and pharmaceutically acceptable salts or solvates of (2);
wherein said method comprises growing a fungus of the genus Penicillium and isolating said compound from the culture medium and/or the fungal biomass.

27. The method according to claim 26, characterised in that the growing of the fungus takes place in a marine organism.

28. The method according to claim 26, further comprising a subsequent synthetic derivatisation of the isolated compound.

29. A method for the biomimetic synthesis of a compound of the general formula (2): 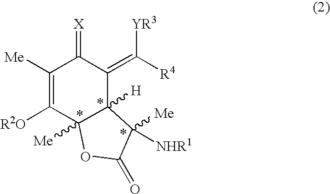
wherein
R¹ is selected from the group consisting of: —H, (C₁-C₁₀)-alkyl (wherein alkyl is straight or branched), (C₃-C₁₀)-alkenyl, and acyl groups, wherein free —COOH-groups can be present on the acyl group in the form of esters; or, optionally, R¹ can be (3) or (4) 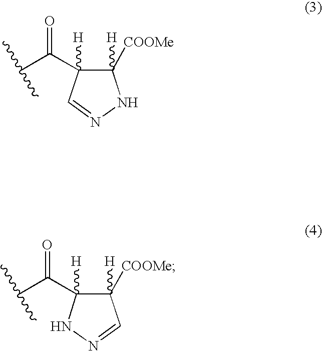
R² is selected from the group consisting of: —H, (C₁-C₁₀)-alkyl (wherein alkyl is straight or branched), and acyl groups;
R³ is selected from the group consisting of: —H, (C₁-C₁₀)-alkyl (wherein alkyl is straight or branched), and acyl groups;
R⁴ is selected from the group consisting of: (C₁-C₁₀)-alkyl (wherein alkyl is straight or branched), and (C₃-C₁₀)-alkenyl, wherein the alkenyl residue can contain one or more double bonds;
X is selected from the group consistinf of O, S, NOH and NOR⁵, wherein R⁵ is a straight chain or branched chain (C₁-C₆)-alkyl;
Y is O, or Y and X are N-atoms bound to each other thus forming a pyrazole ring;
wherein the compound can be present as an (R,R,R)-, (R,R,S)-, (R,S,R)-, (R,S,S)-, (S,R,R)-, (S,R,S)-, (S,S,R)- or (S,S,S)-stereoisomer; and pharmaceutically acceptable salts or solvates of (2);
wherein said method comprises:
a) providing sorbicillin and/or a derivative thereof;
b) oxidative dearomatisation and subsequent addition of alanin or other amino acid or an analogue thereof; and
c) subsequent attachment of fumaric acid or an analogous acyl residue.

30. A pharmaceutical composition comprising a compound of the general formula (2): 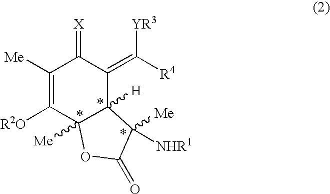
wherein
R¹ is selected from the group consisting of: —H, (C₁-C₁₀)-alkyl (wherein alkyl is straight or branched), (C₃-C₁₀)-alkenyl, and acyl groups, wherein free —COOH-groups can be present on the acyl group in the form of esters; or, optionally, R¹ can be (3) or (4) 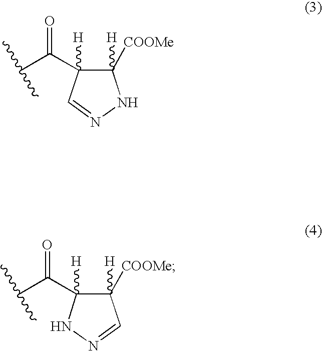
R² is selected from the group consisting of: —H, (C₁-C₁₀)-alkyl (wherein alkyl is straight or branched), and acyl groups;
R³ is selected from the group consisting of: —H, (C₁-C₁₀)-alkyl (wherein alkyl is straight or branched), and acyl groups;
R⁴ is selected from the group consisting of: (C₁-C₁₀)-alkyl (wherein alkyl is straight or branched), and (C₃-C₁₀)-alkenyl, wherein the alkenyl residue can contain one or more double bonds;
X is selected from the group consistinf of O, S, NOH and NOR⁵, wherein R⁵ is a straight chain or branched chain (C₁-C₆)-alkyl;
Y is O, or Y and X are N-atoms bound to each other thus forming a pyrazole ring;
wherein the compound can be present as an (R,R,R)-, (R,R,S)-, (R,S,R)-, (R,S,S)-, (S,R,R)-, (S,R,S)-, (S,S,R)- or (S,S,S)-stereoisomer; and pharmaceutically acceptable salts or solvates of (2);
together with one or more suitable excipients and additives.

31. The pharmaceutical composition according to claim 30, characterised in that the compound is present in the form of a depot substance or as a precursor, together with a suitable, pharmaceutically acceptable diluent or carrier substance.

32. The pharmaceutical composition according to claim 30, characterised in that the compound is present in an amount of 20 μg.

33. The pharmaceutical composition according to claim 30, characterised in that the compound is present in an amount such that a concentration range of between 0.3 and 3.0 μg/ml is present at a treatment in vivo.

34. The pharmaceutical composition according to claim 30, characterised in that it contains further chemotherapeuticals.

35. The pharmaceutical composition according to claim 30, in the form of tablets, dragées, capsules, droplets, suppositories, preparations for injection or infusion for peroral, rectal or parenteral use.

36. A method for the treatment of a disease selected from the group consisting of tumours, viral diseases, and inflammatory conditions, wherein said method comprises administering a compound of the general formula (2): 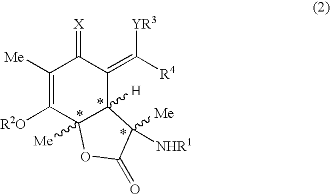
wherein
R¹ is selected from the group consisting of: —H, (C₁-C₁₀)-alkyl (wherein alkyl is straight or branched), (C₃-C₁₀)-alkenyl, and acyl groups, wherein free —COOH-groups can be present on the acyl group in the form of esters; or, optionally, R¹ can be (3) or (4) 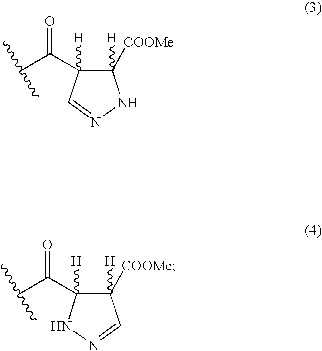
R² is selected from the group consisting of: —H, (C₁-C₁₀)-alkyl (wherein alkyl is straight or branched), and acyl groups;
R³ is selected from the group consisting of: —H, (C₁-C₁₀)-alkyl (wherein alkyl is straight or branched), and acyl groups;
R⁴ is selected from the group consisting of: (C₁-C₁₀)-alkyl (wherein alkyl is straight or branched), and (C₃-C₁₀)-alkenyl, wherein the alkenyl residue can contain one or more double bonds;
X is selected from the group consistinf of O, S, NOH and NOR⁵, wherein R⁵ is a straight chain or branched chain (C₁-C₆)-alkyl;
Y is O, or Y and X are N-atoms bound to each other thus forming a pyrazole ring;
wherein the compound can be present as an (R,R,R)-, (R,R,S)-, (R,S,R)-, (R,S,S)-, (S,R,R)-, (S,R,S)-, (S,S,R)- or (S,S,S)-stereoisomer; and pharmaceutically acceptable salts or solvates of (2).

37. The method according to claim 36, comprising administering the compound in the form of a depot substance or as a precursor, together with a suitable, pharmaceutically acceptable diluent or carrier substance.

38. The method according to claim 36, wherein the viral disease is HIV-1, and the compound is administered in a concentration range of between 0.3 and 3.0 μg/ml.

39. The method according to claim 36, wherein an inflammation is treated, and the compound is administered in a concentration of 20 μg/ml.

40. The method according to claim 36, wherein the formation of oedema is treated, and the compound is administered in an amount of 20 μg.
